(19) United States
(12) Patent Application Publication
(10) Pub. No.: US 2005/0288639 A1
(43) Pub. Date: Dec. 29, 2005

(54) Instrument used in treatment of the urinary incontinence in women

(76) Inventor: Michael Cezary Hibner, Phoenix, AZ (US)

Correspondence Address: Michael C. Hibner, 22226 N. 41st Street, Phoenix, AZ, 85050, US

(21) Appl. No.: 11/158,196

(22) Filed: Jun. 22, 2005

Related U.S. Application Data
(60) Provisional application No. 60/582,631, filed on Jun. 25, 2004.

(51) Int. Cl. A61F 2/00; A61M 29/00; A61M 25/00

(52) U.S. Cl. 604/264; 600/29; 604/96.01

(57) ABSTRACT

A disposable, inexpensive instrument is provided with the aim to facilitate an injection of bulk enhancing agent into the urethral sphincter. An idea of a balloon of Foley's catheter has been exploited where the balloon acts as a retainer immobilizing the instrument in a desired position inside the bladder during the injection. The instrument has a shape of an elongated shaft with a multitude of curved channels guiding and deflecting the needle to an appropriate angle, depth of penetration and exact location on the sphincter during the injection.

### CROSS REFERENCE TO RELATED APPLICATIONS

This application claims the benefit of PPA Ser. No. 60/582,631 filed Jun. 25, 2004 by Michael C. Hibner

### FEDERALLY SPONSORED RESEARCH

Not applicable

### SEQUENCE LISTING

None

### FIELD OF THE INVENTION

This invention relates to instruments and methods used in treatment of the urinary incontinence in women, specifically to the methods in which a fluid bulk enhancing agents like collagen are injected to the tissue of the urethral sphincter.

### BACKGROUNG OF THE INVENTION

Urinary incontinence is a widespread problem throughout the world. Urinary incontinence affects people of all ages and can severely impact a patient, both physiologically and psychologically.

One form of urinary incontinence suffered by women is intrinsic sphincteric deficiency, a condition in which the urethral sphincter does not function properly, thus preventing proper coaptation of the urethra. That kind of deficiency can arise primarily from loss of urethral muscle as well as loss of uretheral vasculature, thinning of urethral mucosa and loss of connective tissue support.

Commonly known approaches to treating urinary incontinence in women require invasive surgical procedures done either through the vaginal or abdominal approach. Most of these methods use various kinds of slings, implants or artificial urinary sphincters.

There are also non-invasive methods and devices depending on insertion of various kinds of devices called pessaries into the patient's vagina, some of them inflatable exerting pressure against the urethra and urethro-vesical junction. These devices and pessaries are rather cumbersome and awkward in use. Also, methods were developed, for example U.S. Pat. No. 6,776,779 and U.S. Pat. No. 6,836,688 depending on delivering to patients pelvic support an electric energy to induce resistive heating selectively contracting or shrinking of the tissue. There are also known methods of implanting expanded Polytetrafluoroethylene (PTFE) tubing delivered into the periurethral tissue to augment a natural contraction of the urethral sphincters. U.S. Pat. No. 6,840,899 describes such method and an instrument for treatment of urinary incontinence delivering implantable material in vivo. The material is in a form of a tube with outside diameter approx. 2.4 mm made of expanded PTFE. The instrument comprises a body with a handle and a reciprocating mechanism pushing the tube through a cannula ended like a trocar into the tissue of the urethra. The procedure has to be done under visual or other forms of control.

Another approach to treating urinary incontinence is an injection of biocompatible bulk-enhancing agents. Such injections can be applied either transurethrally or periurethrally. The method has an advantage of being a less invasive form of treatment and, thus, can be performed on an outpatient basis. The bulk-enhancing agents such as Teflon, autologous fat or collagen increase pressure on urethra and reduce the size of the urethral lumen, providing additional resistance to the flow of urine. U.S. Pat. No. 6,702,731 describes a biocompatible tissue-reactive polymers recently developed. They differ from inert polymers in that they bond with tissue to form a bulk polymer in-situ which is biocompatible, elastometric and non-biodegradable. The bonded polymer does not substantially change volume with time and does not migrate to distant organs within body.

Nowadays, periurethral and transurethral injections are done under direct visualization with the cystoscope. This requires use of expensive cystoscopic equipement, use of the operating room or procedure room and use of anesthesia. Also, physicians doing the procedure must undergo extensive training on how to precisely inject bulking agent with cystoscopic visualization. Anatomically all the injections are made in the area called urethro-vesical junction, which is where urethra is attached to the urinary bladder. This area contains urethral sphincter muscle responsible for the continence in female patients.

My invention relates to injections of bulk enhancing agents applied transurethrally. The idea for the invention had been borne during my fellowship in the Department of Gynecologic Surgery at Mayo Clinic Scottsdale. Although the procedure of applying an injection to the sphincter of the urethra seems to be quite straightforward and easy at first glance, it is not always so. To correctly establish position of the needle's tip inside the urethra I imagined an instrument similar to Foley's catheter with an inflatable balloon at the proximal end. Like in an original Foley's catheter the balloon inserted into the bladder and inflated with water (saline) serves as a locking device, retaining the instrument in the desired position against the sphincter. Yet, the similarity to Foley's catheter ends there since the instrument is not a catheter in the sense that it's function is not to drain the bladder but only to serve as a guide and gauging device for the needle during the injection procedure. Also, materials used are quite different. Instead of soft latex rubber or similar material the instrument will be made of some kind of very hard but inexpensive material of medical grade like MF resin or other suitable material. Unlike Foley's catheter which may stay inside the urethra for days if not weeks, this instrument will be used for a couple of minutes only and then disposed of. Based on the above premises the objectives for my invention were as follows:
a) to provide a disposable instrument which would assist in transurethral injection of bulk-enhancing agents;
b) to provide an instrument which would be reliable yet simple and easy to use even by a physician not trained in the field of urogynecology;
c) to provide an instrument which could be used in the physicians office without the need of an operating room, anesthesia or use of cystoscopic equipment;
d) to provide an instrument which could be used both for applying a bulk enhancing agent and for carrying out a local anesthesia before the main procedure while keeping the instrument in place all time.
e) to provide an instrument which would be inexpensive and sold as a package containing, besides the instrument itself, a bulking agent, analgetic for local anesthesia, needles of predetermined length, syringes including a calibrated syringe to feed an appropriate volume of water into the balloon, a detailed instruction how to use the said instrument, etc.

### SUMMARY OF THE INVENTION

In accordance with the present invention an instrument is proposed utilizing the main thought of Foley's catheter namely the inflatable balloon. The instrument provides means of reliably establishing the correct position of the needle during the injection of bulk-enhancing agent into the urethro-vesical junction and urethral sphincter.

### BRIEF DESCRIPTION OF THE DRAWINGS

FIG. 1 shows a side view of the instrument.

FIG. 2 shows a back view with a partial cross-section of the stem

FIG. 3 shows a side view with a partial cross-section of the stem.

FIG. 4 shows a front view of the stem.

FIG. 5 shows a back view of the nozzle.

FIG. 6 shows a side view and partial cross-section of the nozzle.

FIG. 7 shows a front view of the nozzle.

FIG. 8 shows an exploded perspective view of the stem and the nozzle.

FIG. 9 is an assembly drawing of the instrument.

FIG. 10 is an enlarged view of the detail from FIG. 9.

FIG. 11 is a simplified anatomical sketch to show a mode of application of the instrument.

### DETAILED DESCIPTION OF THE INVENTION

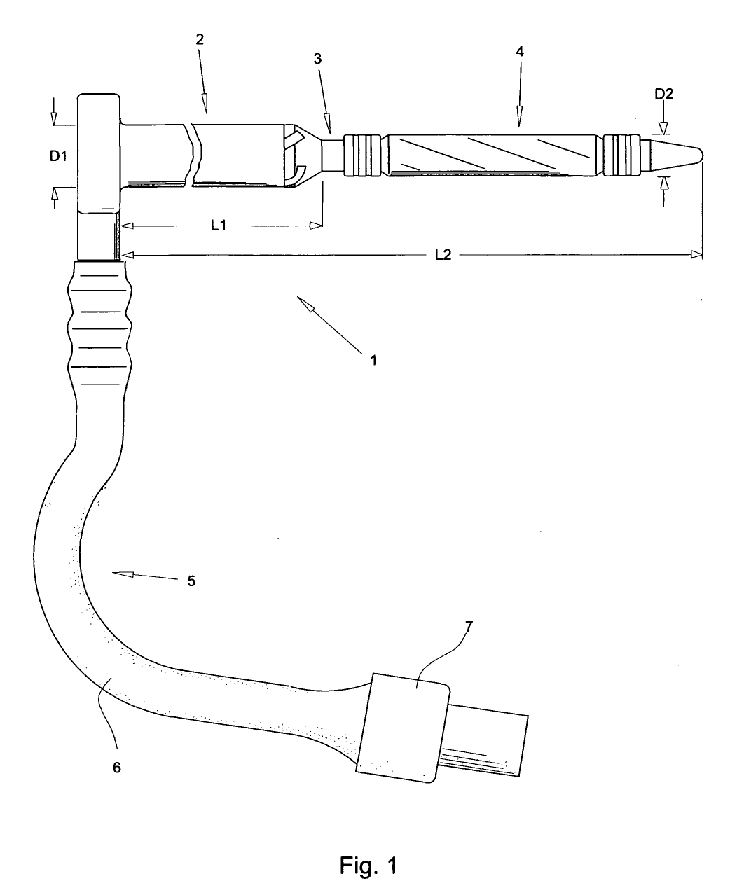

Referring to FIG. 1 an instrument 1 according to my invention is shown composed of four main parts, stem 2, nozzle 3, balloon 4 and water supply means 5. The water supply means like in a typical Foley's catheter comprises a short piece of flexible hose 6 and valve 7. Preferably, diameter D1 of the stem is about 6 mm or 18 French. Length L1 sufficient to pass the female urethra will be about 80 mm making the total active length L2 equal to about 112 mm. The overall diameter D2 of the proximal end of the instrument with the balloon deflated is about 4 mm or 12 French.

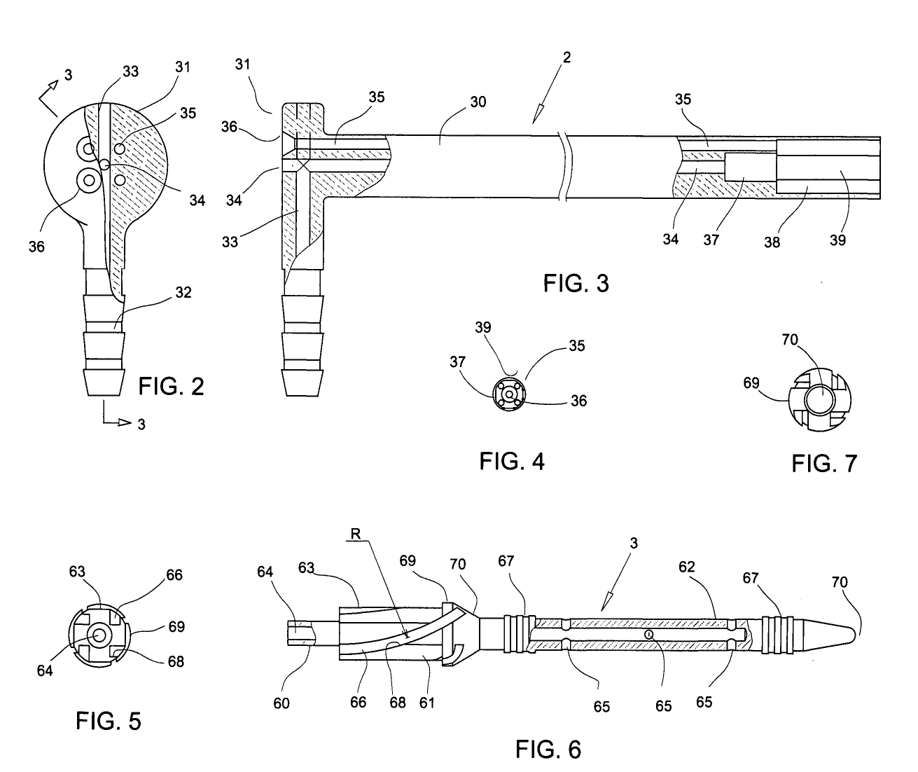

Stem 2 is shown in more detail in FIGS. 2, 3 and 4. It can be made of any type medical grade plastic material suitable for adhesive bonding. The stem is shaped like a long cylinder 30 with flange 31 at its distal end furnished with connector 32 to receive a flexible hose. As can be seen from the drawings, hole 33 is made through the connector and the flange at the right angle to the longitudinal axis of the stem crossing with hole 34 passing centrally through the stem. These two holes deliver water to the balloon. Also, there is a multitude of holes 35 arranged radially with reference to the long axis passing through the stem. In the preferred embodiment there are four such holes and their function is to guide the injection needle. Cones 36 at the outer outlet of the holes 35 serve to provide easier access to an injection needle.

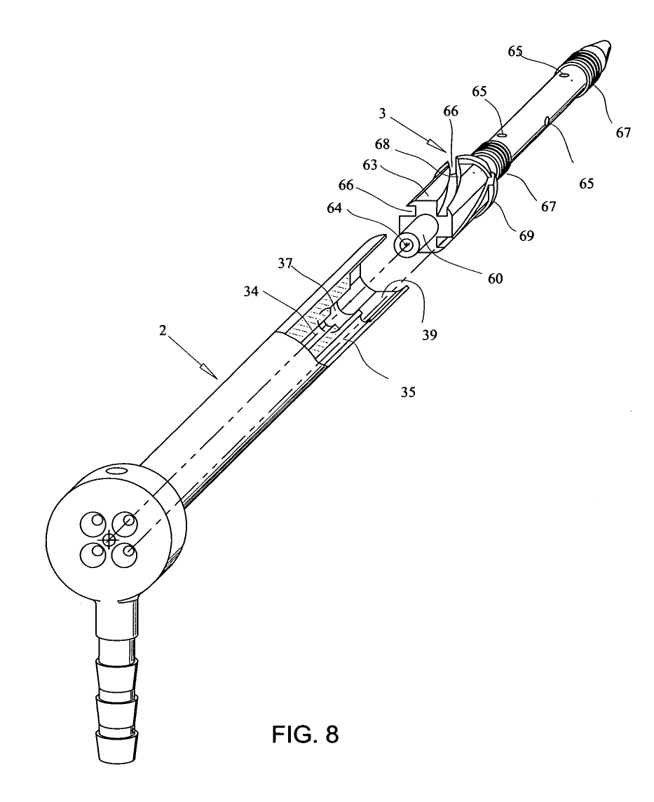

Nozzle 3 is depicted in FIGS. 5, 6 and 7. The nozzle will be made of hard, thermal setting plastic material or cast metal alloy. It has a short sleeve 60 at its back end, barrel 61 in the middle and shaft 62 at the front. Four radially disposed flats 63 on barrel 61 match corresponding flats 39 inside hole 38 of stem 2 as shown in FIGS. 3 and 4. There is a loose fit between sleeve 60 and hole 37 inside the stem. Hole 64 is running through nearly full length of the nozzle with three pairs of transverse holes 65 in shaft 62. When mounted together, holes 65 receive water through hole 64 and hole 34 of the stem. The purpose of providing three pairs of holes 65 is to prevent obstruction of the water outflow when emptying the balloon after the procedure. Key features of barrel 61 are a multitude of radially disposed channels 66. As can be seen from FIGS. 5, 6 and 7 these channels are square in their cross-section and are curved in planes parallel to the long axis of the instrument. Radius R of curvature of these channels is about 20 mm and their number corresponds with the number of holes 35 in the stem. When stem 2 and nozzle 3 are mounted together channels 66 are lying on the extension of holes 35. Their exit is on cone 70 forming a transition surface between flange 69 of barrel 61 and shaft 62. The whole setup is shown on the exploded perspective view in FIG. 8. When an injection needle enters channel 66 it deflects to an angle of about 30-40 degrees to the axis of the instrument. It is especially important for a smooth passage of the deflecting needle that surfaces 68 of channels 66 are made hard. It is therefore possible to produce the nozzle of a softer material with metal inserts embedded into the channel. There are two grooved bosses 67 where the balloon 4 is to be sealed. At its front end the shaft is narrowing conically to end like a sphere 70 to promote travel of the instrument through the urethra.

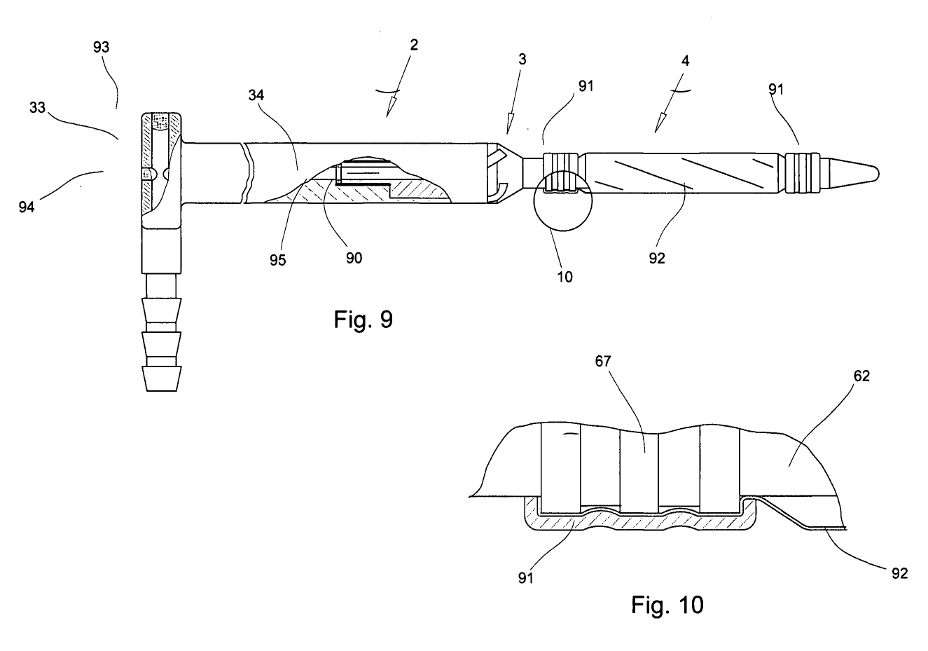

FIGS. 9 and 10 show how the main parts of the instrument are assembled. Stem 2 and nozzle 3 are joined together at point 90 with an adhesive. Balloon 4 consists of an envelope 92 and band clip 91 with which it is fastened to bosses 67 on shaft 62 of the nozzle. Band clip 91 is a piece of a heat shrinking tube which when heated crimps ends of the balloon's envelope 92 on grooves of boss 67. Since the instrument will be used for a very short period of time only, the envelope of the balloon can be made of some cheaper type of material than latex rubber, for example a polyethylene, in which case different methods could be devised for attaching the envelope of the balloon to the nozzle for instance by heat shrinking these two parts directly together. Holes 33 and 34 have to be blocked at points 93 and 94 respectively with an adhesive or some kind of a putty. These holes are open at both ends for technological purposes only. Yet, it is advisable to have the hole 34 passable and open during joining stem 2 and nozzle 3 together. A piece of wire can be put into the hole beyond the point of junction 95 to prevent an excess of adhesive applied to clog the passage.

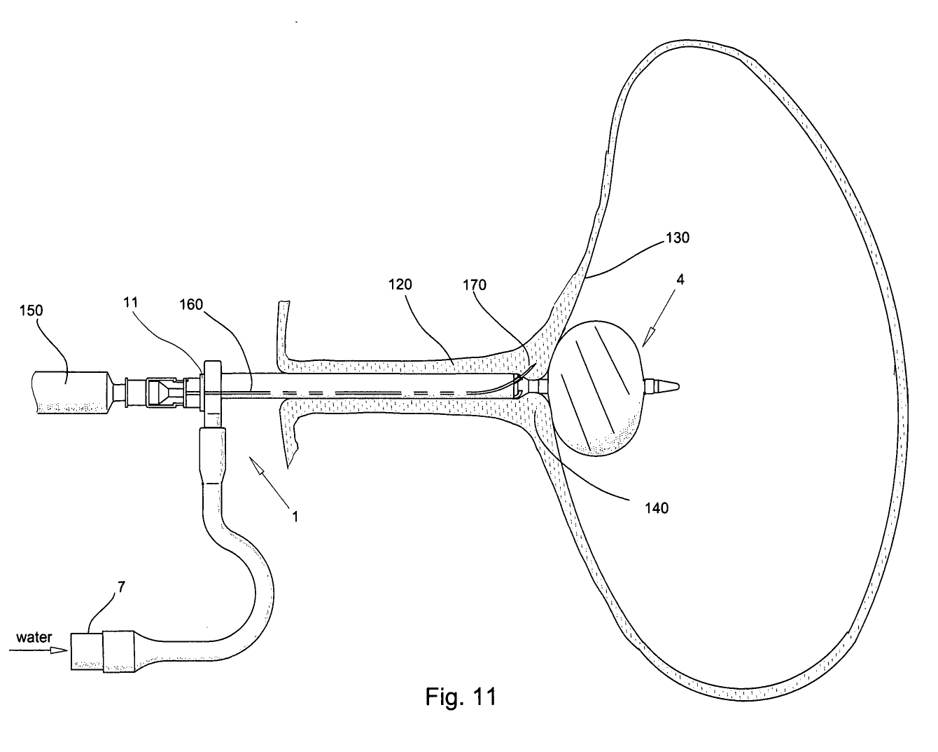

FIG. 11 shows the instrument 1 applied in situ during the injection. The instrument is inserted into urethra 120, deep enough for balloon 4 to be contained fully inside bladder 130. A predetermined volume of water is supplied through valve 7 to inflate the balloon with a syringe not shown in the drawing. The instrument is then slightly pulled backwards so that balloon 4 makes contact with sphincter 140 of the urethra and a bulk-enhancing agent is injected using syringe 150 with needle 160. Needle 160 should be of exactly predefined length so that its deflected front end is entering the tissue of sphincter 170 to a proper depth. Four injections can be applied radially at equal angles of 90 degrees without changing position of the instrument. By rotating the instrument about its axis more injections can be made at other angles, would such need arise. Also, in another embodiment, the instrument can have less than 4 holes to guide the needle, for example 3, 2 or even 1, in which case rotating of the instrument after each injection would be necessary. In yet another embodiment of the instrument the channels deflecting the needle could be made different both as regards their curvature and point of exit so that injections could be made in different areas of the sphincter. In case the injection needle was too long or different depths of needle penetration were desired a set of small flat space washers 11 will be provided together with the instrument.

1. An instrument for injecting a bulk enhancing agent into the sphincter of urethra comprising:
(a) an elongated cylindrical body having a distal end and a proximal end terminating with a flange at said distal end, said body narrowing further on approx ⅔ of its length from said distal end to a spherically ended nozzle at said proximal end, said nozzle having transverse holes connected to a hole running longitudinally to said body, said hole terminating at an outlet connector on said flange, said body including further a multitude of channels starting at a facial plane of said flange and running longitudinally inside said body, said channels being radially curved at an exit in the vicinity of a transition cone where said body is narrowing down to said nozzle,
(b) a balloon of a predetermined size made of a soft and flexible material, enveloping said nozzle approx. in its middle and sealed to bosses on said nozzle at a predetermined distance to said exit of said channels on said body, said transverse holes connected with an interior of said balloon,
(c) a flexible hose of predetermined length terminating at one end with a valve to receive a nozzle of a syringe another end of said hose being connected to said outlet on said flange,
whereby said balloon filled with water through said valve on said flexible hose acts as a retainer for said instrument inside a bladder of a patient, positioning and immobilizing said instrument inserted into said sphincter of urethra, during which process an injection needle introduced into said channel and further deflected on said exit by the curvature of said channel enters a tissue of said sphincter at a correct location and to a correct depth and angle during injection of said bulk enhancing agent.

2. The instrument of claim 1 wherein at least one said channel is provided.

3. The instrument of claim 1 wherein said channels on said instrument assume different shapes and sizes

4. The instrument of claim 1 wherein said nozzle is manufactured as a separate part joined to said distal part of said body by an adhesive.

5. The instrument of claim 1 wherein a set of space washers is provided to adjust: a depth of penetration of said injection needle.
